SEQUENCE LISTING (34 sequences)

SEQ ID NO: 1 - protein, 303 aa, Homo sapiens
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 2 - protein, 227 aa, Homo sapiens
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 3 - protein, 151 aa, Artificial Sequence
  Fragment PILR alpha I derived from paired immunoglobulin-like receptor alpha
MGRPLLLPLLPLLLPPAFLQPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQ

SEQ ID NO: 4 - protein, 85 aa, Artificial Sequence
  Fragment PILR alpha II derived from paired immunoglobulin-like receptor alpha
VRISWRRGHFHGQSFYSTRPPSIHKDYVNRLFLNWTEGQKSGFLRISNLQKQDQSVYFCRVELDTRSSGRQQWQSIEGTKLSITQ

SEQ ID NO: 5 - protein, 32 aa, Artificial Sequence
  Fragment PILR alpha III derived from paired immunoglobulin-like receptor alpha
QSVYFCRVELDTRSSGRQQWQSIEGTKLSITQ

SEQ ID NO: 6 - protein, 53 aa, Artificial Sequence
  Fragment PILR alpha IV derived from paired immunoglobulin-like receptor alpha
VRISWRRGHFHGQSFYSTRPPSIHKDYVNRLFLNWTEGQKSGFLRISNLQKQD

SEQ ID NO: 7 - protein, 16 aa, Artificial Sequence
  Fragment PILR alpha V derived from paired immunoglobulin-like receptor alpha
QSVYFCRVELDTRSSG

SEQ ID NO: 8 - protein, 10 aa, Artificial Sequence
  Fragment PILR alpha VI derived from paired immunoglobulin-like receptor alpha or fragment PILR beta VI derived from paired immunoglobulin-like receptor beta
QSVYFCRVEL

SEQ ID NO: 9 - protein, 7 aa, Artificial Sequence
  Fragment PILR alpha VII derived from paired immunoglobulin-like receptor alpha or fragment PILR beta VII derived from paired immunoglobulin-like receptor beta
YFCRVEL

SEQ ID NO: 10 - protein, 5 aa, Artificial Sequence
  Fragment PILR alpha VIII derived from paired immunoglobulin-like receptor alpha or fragment PILR beta VIII derived from paired immunoglobulin-like receptor beta
YFCRV

SEQ ID NO: 11 - protein, 4 aa, Artificial Sequence
  Fragment PILR alpha IX derived from paired immunoglobulin-like receptor alpha or fragment PILR beta IX derived from paired immunoglobulin-like receptor beta
YFCR

SEQ ID NO: 12 - protein, 151 aa, Artificial Sequence
  Fragment PILR beta I derived from paired immunoglobulin-like receptor beta
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQ

SEQ ID NO: 13 - protein, 85 aa, Artificial Sequence
  Fragment PILR beta II derived from paired immunoglobulin-like receptor beta
VRISWRRGHFHGQSFYSTRPPSIHKDYVNRLFLNWTEGQESGFLRISNLRKEDQSVYFCRVELDTRRSGRQQLQSIKGTKLTITQ

SEQ ID NO: 14 - protein, 32 aa, Artificial Sequence
  Fragment PILR beta III derived from paired immunoglobulin-like receptor beta
QSVYFCRVELDTRRSGRQQLQSIKGTKLTITQ

SEQ ID NO: 15 - protein, 53 aa, Artificial Sequence
  Fragment PILR beta IV derived from paired immunoglobulin-like receptor beta
VRISWRRGHFHGQSFYSTRPPSIHKDYVNRLFLNWTEGQESGFLRISNLRKED

SEQ ID NO: 16 - protein, 16 aa, Artificial Sequence
  Fragment PILR beta V derived from paired immunoglobulin-like receptor beta
QSVYFCRVELDTRRSG

SEQ ID NO: 17 - protein, 6 aa, Artificial Sequence
  Fragment of poly-His region of the synthetic fusion protein
HHHHHH

SEQ ID NO: 18 - protein, 330 aa, Artificial Sequence
  Fc fragment
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 453 bp, Artificial Sequence
  Fragment encoding PILR alpha I derived from paired immunoglobulin-like receptor alpha
atgggtcggcccctgctgctgcccctactgcccttgctgctgccgccagcatttctgcagcctagtggctccacaggatctggtccaagctacctttatggggtcactcaaccaaaacacctctcagcctccatgggtggctctgtggaaatccccttctccttctattacccctgggagttagccacagctcccgacgtgagaatatcctggagacggggccacttccacaggcagtccttctacagcacaaggccgccttccattcacaaggattatgtgaaccggctctttctgaactggacagagggtcagaagagcggcttcctcaggatctccaacctgcagaagcaggaccagtctgtgtatttctgccgagttgagctggacacacggagctcagggaggcagcagtggcagtccatcgaggggaccaaactctccatcacccag

SEQ ID NO: 20 - DNA, 255 bp, Artificial Sequence
  Fragment encoding PILR alpha II derived from paired immunoglobulin-like receptor alpha
gtgagaatatcctggagacggggccacttccacaggcagtccttctacagcacaaggccgccttccattcacaaggattatgtgaaccggctctttctgaactggacagagggtcagaagagcggcttcctcaggatctccaacctgcagaagcaggaccagtctgtgtatttctgccgagttgagctggacacacggagctcagggaggcagcagtggcagtccatcgaggggaccaaactctccatcacccag

SEQ ID NO: 21 - DNA, 96 bp, Artificial Sequence
  Fragment encoding PILR alpha III derived from paired immunoglobulin-like receptor alpha
cagtctgtgtatttctgccgagttgagctggacacacggagctcagggaggcagcagtggcagtccatcgaggggaccaaactctccatcacccag

SEQ ID NO: 22 - DNA, 159 bp, Artificial Sequence
  Fragment encoding PILR alpha IV derived from paired immunoglobulin-like receptor alpha
gtgagaatatcctggagacggggccacttccacaggcagtccttctacagcacaaggccgccttccattcacaaggattatgtgaaccggctctttctgaactggacagagggtcagaagagcggcttcctcaggatctccaacctgcagaagcaggac

SEQ ID NO: 23 - DNA, 48 bp, Artificial Sequence
  Fragment encoding PILR alpha V derived from paired immunoglobulin-like receptor alpha
cagtctgtgtatttctgccgagttgagctggacacacggagctcaggg

SEQ ID NO: 24 - DNA, 30 bp, Artificial Sequence
  Fragment encoding PILR alpha VI derived from paired immunoglobulin-like receptor alpha or fragment encoding PILR beta VI derived from paired immunoglobulin-like receptor beta
cagtctgtgtatttctgccgagttgagctg

SEQ ID NO: 25 - DNA, 21 bp, Artificial Sequence
  Fragment encoding PILR alpha VII derived from paired immunoglobulin-like receptor alpha or fragment encoding PILR beta VII derived from paired immunoglobulin-like receptor beta
tatttctgccgagttgagctg

SEQ ID NO: 26 - DNA, 15 bp, Artificial Sequence
  Fragment encoding PILR alpha VIII derived from paired immunoglobulin-like receptor alpha or fragment encoding PILR beta VIII derived from paired immunoglobulin-like receptor beta
tatttctgccgagtt

SEQ ID NO: 27 - DNA, 12 bp, Artificial Sequence
  Fragment encoding PILR alpha IX derived from paired immunoglobulin-like receptor alpha or fragment encoding PILR beta IX derived from paired immunoglobulin-like receptor beta
tatttctgccga

SEQ ID NO: 28 - DNA, 453 bp, Artificial Sequence
  Fragment encoding PILR beta I derived from paired immunoglobulin-like receptor beta
atgggtcggcccctgctgctgcccctgctgctcctgctgcagccgccagcatttctgcagcctggtggctccacaggatctggtccaagctacctttatggggtcactcaaccaaaacacctctcagcctccatgggtggctctgtggaaatccccttctccttctattacccctgggagttagccatagttcccaacgtgagaatatcctggagacggggccacttccacgggcagtccttctacagcacaaggccgccttccattcacaaggattatgtgaaccggctctttctgaactggacagagggtcaggagagcggcttcctcaggatctcaaacctgcggaaggaggaccagtctgtgtatttctgccgagtcgagctggacacccggagatcagggaggcagcagttgcagtccatcaaggggaccaaactcaccatcacccag

SEQ ID NO: 29 - DNA, 255 bp, Artificial Sequence
  Fragment encoding PILR beta II derived from paired immunoglobulin-like receptor beta
gtgagaatatcctggagacggggccacttccacgggcagtccttctacagcacaaggccgccttccattcacaaggattatgtgaaccggctctttctgaactggacagagggtcaggagagcggcttcctcaggatctcaaacctgcggaaggaggaccagtctgtgtatttctgccgagtcgagctggacacccggagatcagggaggcagcagttgcagtccatcaaggggaccaaactcaccatcacccag

SEQ ID NO: 30 - DNA, 96 bp, Artificial Sequence
  Fragment encoding PILR beta III derived from paired immunoglobulin-like receptor beta
cagtctgtgtatttctgccgagtcgagctggacacccggagatcagggaggcagcagttgcagtccatcaaggggaccaaactcaccatcacccag

SEQ ID NO: 31 - DNA, 159 bp, Artificial Sequence
  Fragment encoding PILR beta IV derived from paired immunoglobulin-like receptor beta
gtgagaatatcctggagacggggccacttccacgggcagtccttctacagcacaaggccgccttccattcacaaggattatgtgaaccggctctttctgaactggacagagggtcaggagagcggcttcctcaggatctcaaacctgcggaaggaggac

SEQ ID NO: 32 - DNA, 48 bp, Artificial Sequence
  Fragment encoding PILR beta V derived from paired immunoglobulin-like receptor beta
cagtctgtgtatttctgccgagtcgagctggacacccggagatcaggg

SEQ ID NO: 33 - DNA, 18 bp, Artificial Sequence
  Fragment encoding poly-His region of synthetic fusion protein
caccaccaccaccaccat

SEQ ID NO: 34 - DNA, 993 bp, Artificial Sequence
  Fc fragment sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

(19) United States
(12) Patent Application Publication
(10) Pub. No.: US 2010/0197576 A1
(43) Pub. Date: Aug. 5, 2010

(54) Polypeptides or fusion proteins thereof inhibiting transmigration of leucocytes or growth and/or metastasis of cancer cells

(75) Inventors: Jang-Hee Hahn, Seoul (KR); Sun-Hee Lee, Wonju-si (KR)

Correspondence Address: THE NATH LAW GROUP, 112 South West Street, Alexandria, VA, 22314, US

(73) Assignee: KNU-INDUSTRY COOPERATION FOUNDATION, Chuncheon-si (KR)

(21) Appl. No.: 12/585,377

(22) Filed: Sep. 14, 2009

(30) Foreign Application Priority Data
Sep. 17, 2008 (KR) 10-2008-0090974

(51) Int. Cl. A61K 38/16; C07K 5/10; C07K 7/06; C07K 7/08; C07K 14/00; C07K 16/00; C07H 21/00; C12N 15/74; C12N 1/21; C12N 1/19; A61K 38/07; A61K 38/08; A61K 38/10; A61P 37/02

(52) U.S. Cl. 514/12; 530/330; 530/329; 530/328; 530/327; 530/326; 530/325; 530/324; 530/350; 530/387.3; 536/23.1; 435/320.1; 435/252.33; 435/254.23; 514/13; 514/14; 514/15; 514/16; 514/17; 514/18

(57) ABSTRACT

The present invention provides a polypeptide or its fusion protein derived from a paired immunoglobulin-like receptor α or β (PILRα or PILRβ), one of the transmembrane proteins, which inhibits the transmigration of leukocytes or the growth and/or metastasis of cancer cells. The present invention also provides a polynucleotide encoding the polypeptide or its fusion protein, a vector including the polynucleotide, and a transformant transformed with the vector. The present invention also provides a pharmaceutical composition for the prevention or treatment of inflammatory diseases including the polypeptide or a fusion protein thereof. The present invention also provides a pharmaceutical composition for inhibiting the growth and/or metastasis of cancer cells including the polypeptide or a fusion protein thereof.

### CROSS-REFERENCE TO RELATED PATENT APPLICATIONS

This application claims the benefit of Korean Patent Application No. 10-2008-0090974, filed on 17 Sep. 2008, in the Korean Intellectual Property Office, the disclosure of which is incorporated herein in its entirety by reference.

### BACKGROUND OF THE INVENTION

1. Field of the Invention

The present invention relates to a polypeptide or its fusion protein derived from a paired immunoglobulin-like receptor α or β (PILRα or PILRβ), one of the transmembrane proteins, which inhibits the transmigration of leukocytes or the growth and/or metastasis of cancer cells. The present invention also relates to a polynucleotide encoding the polypeptide, a vector including the polynucleotide, and a transformant transformed with the vector. The present invention also relates to a pharmaceutical composition for inhibiting the growth and/or metastasis of cancer cells including the polypeptide or a fusion protein thereof. The present invention also relates to a pharmaceutical composition for the prevention or treatment of inflammatory diseases including the polypeptide or a fusion protein thereof.

2. Description of the Related Art

An inflammatory response is known as a protective response of living organism for rehabilitating the structures and functions of tissues damaged by infection, trauma, etc. Mobilization of leukocytes to a focus of inflammation is critical for the rapid resolution of infections and restoration of tissue damages resulting from a variety of injuries. However, a misdirected or prolonged inflammatory response causes damage to the body's tissues or diseases. For example, inflammatory diseases are caused by bacterial or viral infection, e.g., cerebrospinal meningitis, enteritis, dermatitis, uveitis, encephalitis, or adult respiratory distress syndrome, or non-infectious factors, e.g., trauma, autoimmune diseases, or organ transplantation rejection. Inflammatory diseases are classified into acute and chronic inflammatory diseases according to symptoms or pathological features. Acute inflammation such as allergy or bacterial/viral infection is manifested as local signs such as a change in bloodstream, blood vessel size, and vascular permeability, and the recruitment of leukocytes. In contrast, a main pathological feature of chronic inflammation such as rheumatoid arthritis, artherosclerosis, chronic kidney infection, or hepatocirrhosis is a continuous emigration of macrophages, lymphocytes, or plasma cells into foci of inflammation due to recurrence of inflammatory factors, thereby causing a long-lasting inflammatory response.

In order to induce an inflammatory response, the emigration of leukocytes into inflammation foci is an essential event. Many cell adhesion molecules are implicated in the emigration of leukocytes. That is, the emigration of leukocytes includes a rolling stage in which leukocytes are mobilized to the blood vessels of inflamed sites by chemokine secreted from the inflamed sites and then rolled on surfaces of vascular endothelial cells while reducing the velocity of cell movement; an adhesion stage in which the leukocytes stops rolling and are firmly adhered to the vascular endothelial cells; and a transmigration stage wherein the leukocytes migrate through capillary vessels and basement membranes. The final stage, i.e., the transmigration stage is also called “diapedesis” or “transendothelial migration”.

Cancer cells induced by carcinogens proliferate rapidly relative to normal cells, thereby forming tumor masses, invading surrounding tissues, and interfering with normal body functions. Cancer cells bring nutrients and oxygen by inducing angiogenesis, and metastasis thereof is also caused by angiogenesis. Although cancer cells grow infinitely at specific sites, they can also leave the sites from which they originated, migrate to and grow in new sites, whose process is called “metastasis”. Metastasis involve several key steps: conversion of cancer cells to migratory mesenchymal cells, dissociation of the mesenchymal cells from the original tumor sites, invasion into and spread through surrounding connective tissues and capillary vessels, migration through blood vessels, escape from the blood vessels, migration through connective tissues, and proliferation in secondary sites.

Expression and activation of cell adhesion molecules on surfaces of tumor cells play a very important role in tumor metastasis (Zetter, B. R. (1993). Adhesion molecules in tumor metastasis. Semin Cancer Biol. 4: 219). Tumor metastasis is induced by regulating the expression pattern and activity of cell adhesion molecules on surfaces of tumor cells. In order to understand the metastasis of tumor cells, it is prerequisite to understand cell adhesion molecules and substances for regulating the expression and functions of the cell adhesion molecules (Bailly, M., Yan, L., Whitesides, G. M., Condeelis, J. S., and Segall, J. E. (1998). regulation of Protusion Shape and Adhesion to the sustratum during chemoacic esponses of mammalian carcinoma cells. Exp Cell Res. 241: 285; Frisch, S. M., Vuori, K., Ruoslahti, E., and Chan-Hui., P. (1996). Control of adhesion-dependent cell survival by focal adhesion kinase. J Cell Biol 134: 793; and Hannigan, G. E., Leung-Hagesteijn, C., Fitz-Gibbon, L., Coppolino, M. G., Radeva, G., Filmus, J., Bell, J. C., and Dedhar, S. (1996). Regulation of cell adhesion and anchorage-dependent growth by a new β1-integrin-linked protein kinase. Nature 379: 91).

The present inventors have disclosed that when CD99 is activated, the function of βᵢ integrin is altered, thereby preventing the adhesion of cancer cells onto extracellular matrices (ECMs). This suggests that CD99 may be involved in metastasis of cancer cells (Suh JS., 2001. Control of invasiveness of human breast carcinoma cell line MCF-7 by CD99 molecule. Kangwon National University). In addition, the present inventors have disclosed that peptide fragments derived from CD99 can activate CD99, thereby preventing the transmigration of leukocytes or the growth and/or metastasis of cancer cells (International Patent Publication No. WO 2007/037601).

Meanwhile, a paired immunoglobulin-like receptor family (PILR family), known as one of the natural ligands of CD99, is composed of PILRα and PILRβ (Mousseau, D. D., D. Banville, D. L'Abbe, P. Bouchard, and S.-H. Shen. 2000. PILRα, a novel immunoreceptor tyrosine-based inhibitory motif-bearing protein, recruits SHP-1 upon tyrosine phosphorylation and is paired with the truncated counterpart PILRβ. J. Biol. Chem. 275:4467; Fournier, N., L. Chalus, I. Durand, E. Garcia, J. J. Pin, T. Churakova, S. Patel, C. Zlot, D. Gorman, S. Zurawski, et al. 2000. FDF03, a novel inhibitory receptor of the immunoglobulin superfamily, is expressed by human dendritic and myeloid cells. J. Immunol. 165:1197). PILRα and PILRβ are a kind of type I transmembrane proteins, which are composed of the glycosylated extracellular domain, transmembrane region, and the cytoplasmic domain. The amino acid sequences of the extracellular domains of PILRα and PILRβ are highly homologous, while those of the cytoplasmic domains thereof are quite different. The cytoplasmic domain of PILRα has immunoreceptor tyrosine-based inhibitory motifs (ITIMs), which inhibit activity of cells. However, since PILRβ expressed in natural killer cells (NK cells) or dendritic cells does not have ITIMs, it is combined with its ligand to activate the cells, unlike PILRα (Shiratori I., Ogasawara K., Saito T., Lanier LL., Arase H. (2004). Activation of natural killer cells and dendritic cells upon recognition of a novel CD99-like ligand by paired immunoglobulin-like type 2 receptor. J Exp Med 199: 525).

### SUMMARY OF THE INVENTION

The present inventors have found that a polypeptide having a certain amino acid sequence derived from PILRα or PILRβ, natural ligands to CD99, can prevent the transmigration of leukocytes or the growth and/or metastasis of cancer cells. In accordance with an aspect of the present invention, there is provided a polypeptide consisting of 4-200 amino acids derived from the peptide of SEQ ID NOs: 1 or 2, wherein said polypeptide comprising an amino acid sequence from position 123 to position 126 of SEQ ID NOs: 1 or 2; and wherein said polypeptide inhibits transmigration of leukocytes or growth and/or metastasis of cancer cells.

In accordance with another aspect of the present invention, there is provided a fusion protein wherein a polyhistidine (poly-His) region or a Fc region is fused to the polypeptide.

In accordance with still another aspect of the present invention, there is provided a polynucleotide encoding the polypeptide.

In accordance with still another aspect of the present invention, there is provided a vector comprising the polynucleotide encoding the polypeptide.

In accordance with still another aspect of the present invention, there is provided a transformant obtained by transforming a host cell with the vector.

In accordance with yet another aspect of the present invention, there is provided a pharmaceutical composition for inhibiting the growth and/or metastasis of cancer cells, comprising the polypeptide or its fusion protein as an active ingredient and a pharmaceutically acceptable carrier.

In accordance with a further aspect of the present invention, there is provided a pharmaceutical composition for the prevention or treatment of inflammatory diseases, comprising the polypeptide or its fusion protein as an active ingredient and a pharmaceutically acceptable carrier.

### ADVANTAGEOUS EFFECTS

The polypeptide or its fusion protein according to the present invention can inhibit is the transmigration of leukocytes or the growth and/or metastasis of cancer cells. Therefore, the polypeptide or its fusion protein can be applied to a pharmaceutical composition for inhibiting the growth and/or metastasis of cancer cells; and/or for the prevention or treatment of inflammatory diseases.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates the structures of polypeptides as set forth in SEQ ID NOs: 3-16;

FIGS. 2 and 3 are graphs illustrating effects of polypeptides of the present invention on adhesion of human breast carcinoma cells (MCF-7 cells) to fibronectin;

FIG. 4 is a graph illustrating invasion assay for MCF-7 cells after treatment of a polypeptide of the present invention;

FIG. 5 is a graph illustrating transendothelial migration assays for MCF-7 cells after administration of a polypeptide of the present invention;

FIGS. 6 and 7 are graphs illustrating effects of polypeptides of the present invention on adhesion of HUVEC (Human Umbilical Vein Endothelial Cell) to fibronectin;

FIG. 8 is inverted microscopic images showing effects of polypeptides of the present invention on angiogenesis;

FIG. 9 is graphs illustrating effects of polypeptides of the present invention on the transmigration of a human monocyte cell line, U937.

### DETAILED DESCRIPTION OF THE INVENTION

The present invention will now be described more fully with reference to the accompanying drawings, in which exemplary embodiments of the invention are shown.

The polypeptide or its fusion protein according to the present invention can inhibit the transmigration of leukocytes or the growth and/or metastasis of cancer cells. Therefore, the polypeptide or its fusion protein can be applied to a pharmaceutical composition for inhibiting the growth and/or metastasis of cancer cells; and/or for the prevention or treatment of inflammatory diseases.

Throughout the specification, the term “inflammation” or “inflammatory diseases” include acute and/or chronic inflammatory diseases, e.g., rheumatoid arthritis, adhesive capsulitis, sinovitis, coxarthritis, osteoarthritis, osteoporosis, periarthritis, multiple sclerosis, osteomyelitis, systemic lupus erythematosus, polymyalgia rheumatica (PMR), Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroditis, Graves' disease, Goodpasture's disease, mixed connective tissue disease, sclerosing cholangitis; inflammatory bowel disease such as Crohn's Disease or ulcerative colitis, inflammatory dermatoses; inflammatory respiratory diseases such as usual interstitial pneumonitis (UIP), lymphoid interstitial pneumonia, giant cell interstitial pneumonia, cellular interstitial pneumonia, desquamative interstitial pneumonia, asbestosis, silicosis, berylliosis, talcosis, pneumoconiosis, Adult Respiratory Distress Syndrome, extrinsic allergic alveolitis; immediate hypersensitivity reactions such as asthma or hayfever; sarcoidosis, Wegener's granulomatosis, various angiitis, chronic active hepatitis, delayed-type hypersensitivity reactions such as poison ivy dermatitis, cutaneous allergies, psoriatic arthritis, Reiter's syndrome, immediate hypersensitivity reactions, rheumatic fever, acute or chronic glomerulonephritis, acute exacerbations, pyelonephritis, cellulitis, cystitis, acute cholecystitis, inflammatory aortic aneurysm, atherosclerosis, Still's disease), Parkinson's disease, Alzheimer's disease. A polypeptide or fusion protein of the present invention can also be administered to patients suffering from diseases involving inflammatory diseases, e.g., reperfusion injuries, autoimmune diseases, organ transplantation rejection or tissue allograft organ rejection, etc. Thus, the “inflammatory diseases” as used herein are meant to comprehend diseases involving inflammatory diseases. A polypeptide or fusion protein to of the present invention can be used in rheumatoid arthritis, osteoporosis, respiratory inflammation, autoimmune diseases, and/or organ transplantation rejection, particularly preferably, rheumatoid arthritis, autoimmune diseases, and/or organ transplantation rejection.

The present inventors have made various searches for ligand(s) capable of activating the CD99 molecule. In view that PILRα or PILRβ, which are transmembrane protein, acts as a natural ligand recognizing the CD99 molecule, we made ligands of various lengths from PILRα or PILRβ and made search using the ligands. Surprisingly, the present inventors found that polypeptides having the amino acid sequences derived from PILRα or PILRβ could inhibit not only the growth of cancer cells by suppressing tumor angiogenesis, but also their metastasis by inhibiting transendothelial migration of cancer cells through binding to the surfaces of the endothelial cells or cancer cells in high affinity. Those results show that the polypeptides have therapeutic activity against various cancers, especially metastatic cancer. Furthermore, the present inventors found that polypeptides having the amino acid sequences derived from PILRα or PILRβ could show anti-inflammatory activity by blocking the transmigration of leukocytes through high-affinity binding to vascular endothelial cells and leukocytes.

The present invention provides a polypeptide consisting of 4-200 amino acids derived from the peptide of SEQ ID NOs: 1 or 2, wherein said polypeptide comprising an amino acid sequence from position 123 to position 126 of SEQ ID NOs: 1 or 2; and wherein said polypeptide inhibits transmigration of leukocytes or growth and/or metastasis of cancer cells. Preferably, the polypeptide may be selected from the group consisting of polypeptides as set forth in SEQ ID NOs: 3-5, 7-14, and 16.

The present invention also provides a fusion protein wherein a polyhistidine (poly-His) region or a Fc region is fused to the polypeptide. The poly-His region, which is a tag polypeptide, can be used for the separation and purification of the polypeptide of the present invention by binding to a histidine binding resin. In the fusion protein of the present invention, the poly-His region may have an amino acid sequence as set forth in SEQ ID NO: 17. The Fc region can be used for increasing stability in the blood of the polypeptide. In the fusion protein of the present invention, the Fc region may have an amino acid sequence as set forth in SEQ ID NO: 18.

The present invention also provides a polynucleotide encoding the polypeptide. The polynucleotide can be prepared from the nucleic acid sequences encoding PILRα or PILRβ, using a known method in the art. The polynucleotide may have a nucleotide sequence selected from the group consisting of nucleotide sequences as set forth in SEQ ID NOs: 19-21, 23-30, and 32.

The present invention also provides a vector comprising the polynucleotide encoding the polypeptide. Various known cloning vectors, e.g., pPICZα A, B, or C (Invitrogen, U.S.A.), may be used as a cloning vector. Preferably, a vector including DNA encoding a poly-His region, e.g., a pET28a(+) vector (Novagen, U.S.A.) may be used as a cloning vector. And also, a vector obtained by inserting DNA encoding a Fc region (e.g., cDNA consisting of a nucleotide sequence as set forth in SEQ ID NO: 25) into a conventional vector, e.g., a pET28a(+) vector (Novagen, U.S.A.) may be used as a cloning vector. The vector of the present invention can be constructed by inserting the polynucleotide encoding the polypeptide into a cloning vector with an appropriate restriction enzyme site using a method commonly known in the art. The vector of the present invention may be directly used in a gene therapeutic composition for the purpose of gene therapy or may be used in the production of transformants.

The present invention also provides a transformant obtained by transforming a host cell with the vector. The host cell is not particularly limited as long as the polypeptide can be effectively expressed. Preferably, the host cell may be selected from microorganisms belonging to the genus Escherichia (e.g., Escherichia coli), the genus Pichia (e.g., X-33 Pichia; Invitrogen, U.S.A.), etc.

The present invention also provides a pharmaceutical composition for inhibiting the growth and/or metastasis of cancer cells, comprising the polypeptide or its fusion protein as an active ingredient and a pharmaceutically acceptable carrier.

The present invention also provides a pharmaceutical composition for the prevention or treatment of inflammatory diseases, comprising the polypeptide or its fusion protein as an active ingredient and a pharmaceutically acceptable carrier.

The pharmaceutical compositions of the present invention may include excipients such as lactose or corn starch, lubricants such as magnesium stearate, currently available emulsifiers, suspending agents, buffers, isotonic agents, etc. The pharmaceutical compositions of the present invention can be administered orally or parenterally. Preferably, the pharmaceutical compositions of the present invention can be formulated into parenteral dosage forms. For intramuscular, intraperitoneal, subcutaneous, or intravenous administration, a sterilized solution of an active ingredient is generally prepared. In this case, the sterilized solution may include a buffer to is achieve a desired pH value. With respect to formulations for intravenous administration, an isotonic agent may be used to render the formulations isotonic. The pharmaceutical compositions of the present invention can be formulated into aqueous solutions including a pharmaceutically acceptable carrier such as a saline of pH 7.4. The aqueous solutions can be introduced into a patient's intramuscular blood stream by local bolus injection.

The pharmaceutical composition of the present invention can be administered to patients who suffer from various inflammatory diseases, solid cancer (such as breast cancer, gastric cancer, large bowel cancer, colon cancer, rectal cancer, pancreatic cancer) or lymphoma at a daily dosage of about 0.01 to 10 mg/kg. An adequate dosage is generally changed according to age, body weight, and conditions of a patient.

Hereinafter, the present invention will be described more specifically by the following working examples. However, the following working examples are provided only for illustrations and thus the present invention is not limited to or by them.

### Example 1

### Synthesis of Polypeptides

cDNA fragments of SEQ ID NOs: 19-25 and 28-32 encoding respective polypeptides of SEQ ID NOs: 3-9 and 12-16 were inserted into pET28a(+) vectors to produce seven types of pET28a-hPILRα vectors and five types of pET28a-hβ vectors. That is, the cDNA fragments of SEQ ID NOs: 19-25 and 28-32 were isolated by PCR, digested with EcoRI, and inserted into the EcoRI sites of pET28a(+) vectors with ligation enzymes to produce the pET28a-hPILRα vectors and pET28a-hβ vectors. In case of SEQ ID NOs: 3-5 and 12-14, cDNA fragments thereof were inserted into pET28a(+)-Fc vectors, which had been obtained by inserting cDNAs encoding the Fc regions of human immunoglobulin (i.e., cDNA consisting of a nucleotide sequence as set forth in SEQ ID NO: 34) into the pET28a(+) vectors, to produce pET28a-hPILRα I, II, and III-Fc vectors (or pET28a-hPILIRβ I, II, and III-Fc vectors). Colonies obtained by transforming BL21(DE3) cells with the obtained expression vectors were cultured in LB media for about 4-6 hours. When the absorbance (A600) of the cultures reached 0.4-0.6, protein expression was induced by isopropyl 3-D-1-thiogalactopyranoside (IPTG) (1.4 mM) for 7-9 hours. The cells were precipitated by centrifugation, washed with phosphate buffered saline (PBS), and is precipitated to remove impurities from the media. Fractions were analyzed by SDS-PAGE gel to check protein expression.

For purification of expressed proteins, an 8M urea buffer (8M urea, 0.01 M Tris-Cl, 0.1 M NaH₂PO₄) was used. The pH of the urea buffer was adjusted to 8.0, 6.3, 4.5, etc. according to a purification step. The cells were lysed with a pH 8.0 urea buffer containing protease inhibitors (1 mM PMSF, 10 μg/Ml leupeptin, 1 μg/l, pepstatin, 1 μg/Ml aprotinin) and centrifuged at 13,000 rpm for 20 minutes at 4° C. The supernatants were mixed with histidine (His)-binding resins (Ni-NTA His Bind Resins, Novagen, U.S.A.) in a 1 Ml Eppendorf tube, and the mixtures were incubated at 4° C. for 16 hours to induce the binding of histidine residues of the expressed proteins with the His-binding resins. The reaction solutions were centrifuged, the supernatants were discarded, and the pellets were washed with a pH 6.3 urea buffer. The protein was then dialyzed against PBS, and stored in aliquots in a cold store.

Polypeptides of SEQ ID NOs: 10 and 11 were synthesized with an automatic peptide synthesizer (PeptrEx-R48, Peptron, Daejeon, Korea) using a FMOC solid-phase method. The synthesized polypeptides were purified and analyzed by reverse-phase high-performance liquid chromatography (reverse-phase HPLC) (Prominence LC-20AB, Shimadzu, Japan) using a C18 analytical RP column (Shiseido capcell pak), and isolated using a mass spectrometer (HP 1100 Series LC/MSD, Hewlett-Packard, Roseville, U.S.A.).

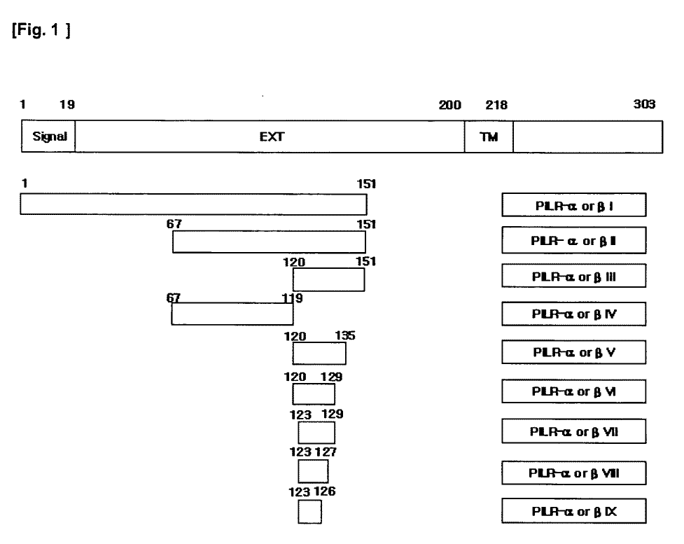

The polypeptides obtained in the above and cDNAs encoding the same are illustrated in the following Table 1 and FIG. 1. As shown in SEQ ID NOs: 1 and 2, the amino acid sequences and nucleic acid sequences of PILRα VI to IX are the same as those of PILRβ VI to IX.

TABLE 1

| | Fragments | Polypeptide | cDNA |
|---|---|---|---|
| PILR α I | 1-151 | SEQ ID NO: 3 | SEQ ID NO: 19 |
| PILR α II | 67-151 | SEQ ID NO: 4 | SEQ ID NO: 20 |
| PILR α III | 120-151 | SEQ ID NO: 5 | SEQ ID NO: 21 |
| PILR α IV | 67-119 | SEQ ID NO: 6 | SEQ ID NO: 22 |
| PILR α V | 120-135 | SEQ ID NO: 7 | SEQ ID NO: 23 |
| PILR α VI | 120-129 | SEQ ID NO: 8 | SEQ ID NO: 24 |
| PILR α VII | 123-129 | SEQ ID NO: 9 | SEQ ID NO: 25 |
| PILR α VIII | 123-127 | SEQ ID NO: 10 | SEQ ID NO: 26 |
| PILR α IX | 123-126 | SEQ ID NO: 11 | SEQ ID NO: 27 |
| PILR β I | 1-151 | SEQ ID NO: 12 | SEQ ID NO: 28 |
| PILR β II | 67-151 | SEQ ID NO: 13 | SEQ ID NO: 29 |
| PILR β III | 120-151 | SEQ ID NO: 14 | SEQ ID NO: 30 |
| PILR β IV | 67-119 | SEQ ID NO: 15 | SEQ ID NO: 31 |
| PILR β V | 120-135 | SEQ ID NO: 16 | SEQ ID NO: 32 |
| PILR β VI | 120-129 | SEQ ID NO: 8 | SEQ ID NO: 24 |
| PILR β VII | 123-129 | SEQ ID NO: 9 | SEQ ID NO: 25 |
| PILR β VIII | 123-127 | SEQ ID NO: 10 | SEQ ID NO: 26 |
| PILR β IX | 123-126 | SEQ ID NO: 11 | SEQ ID NO: 27 |

### Example 2

### Preparation of Polypeptide-Containing Compositions

The polypeptides of SEQ ID NOs: 3-16 were dissolved in PBS to a concentration of 3 μg/100 μl. The resultant protein solutions were used in the following experimental examples.

### Experimental Example 1

### Tests for Inhibitory Activity Against Adhesion of MCF-7 Cells to Extracellular Matrix

Effects of polypeptides of SEQ ID NOs: 3-6 and 9-12 on adhesion of human breast carcinoma cells (MCF-7 cells) to fibronectin were tested.

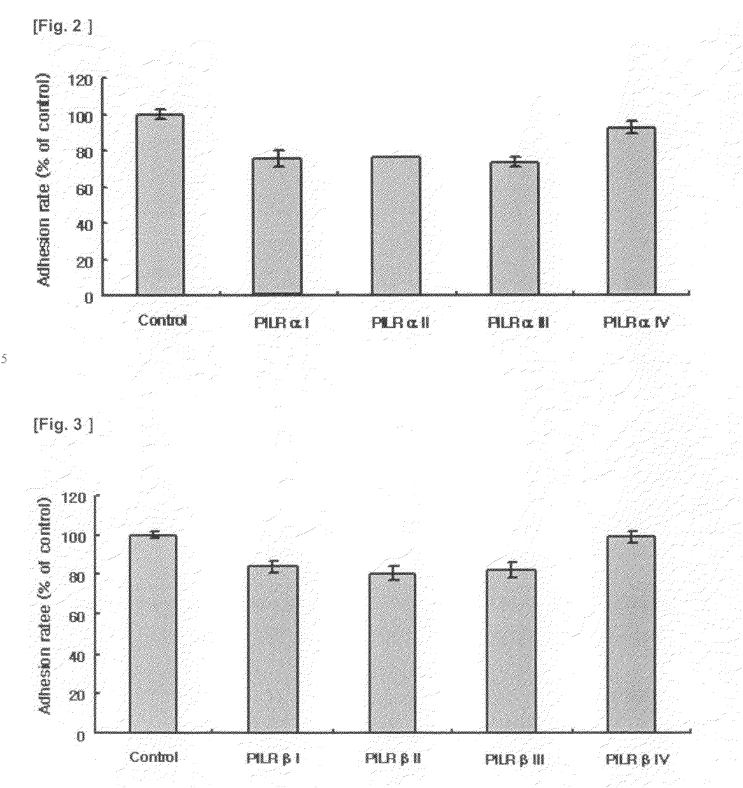

Each well of a 96-well culture plate was streaked with fibronectin, a component of extracellular matrix, and then dried under UV light. MCF-7 cells (5×10⁴) were dispensed into each well, and then the protein solutions including the polypeptide of SEQ ID NOs: 3-6 and 9-12 prepared in Example 2 were treated to each well, in the concentration of 3 μg per each well. After incubation in 5% CO₂ at 37° C. for 1 hour, the cells were washed three times with PBS, detached using trypsin-EDTA, and then stained with a trypan-blue solution. The number of the cells was determined using a hemacytometer. The results are illustrated in FIGS. 2 and 3. In FIGS. 2 and 3, the control peptide is a human IgG Fc, having a polypeptide as set forth in of SEQ ID NO: 18.

Referring to FIGS. 2 and 3, in the test groups treated with the polypeptides of the present invention, the number of MCF-7 cells adhered to fibronectin was reduced by about 10-40% relative to a control group. However, in the test group treated with the polypeptide of SEQ ID NOs: 6 or 12 including no amino acids at positions 123-129 of SEQ ID NOs: 1 and 2, the degree of adhesion of MCF-7 cells to fibronectin was similar to that of the control group.

### Experimental Example 2

### Tests for Inhibitory Activity Against Invasion of Cancer Cells

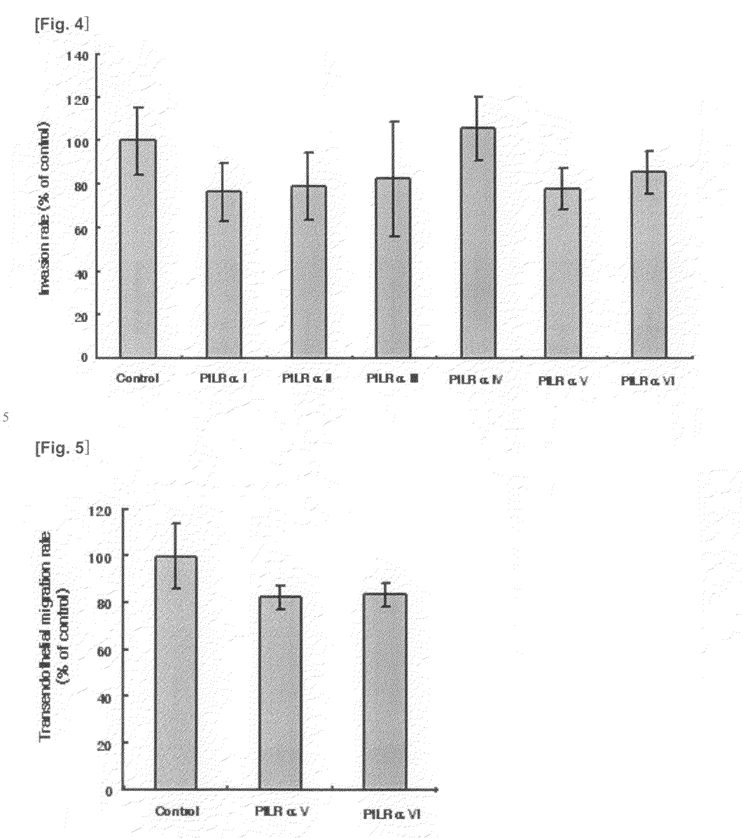

Invasion assay was performed using Transwell chambers (Costar, Cambridge, Mass., USA). Each well was coated with fibronectin, which is a ligand of integrin. MCF-7 cells (5×10⁵ cells) in the serum free media were loaded to the upper compartment of the transwell and then incubated for 24 hours. When about 80% of the cells were grown up, each well was treated with the polypeptides of SEQ ID NOs: 3 to 8, in the concentration of 3 μg per each well. After incubation in 5% CO₂ at 37° C. for 1 hour, each well was treated with 0.1% BSA. Invasion-inducing medium (the supernatant obtained by incubating NIH/3T3 cells (mouse fibroblast cells) in the serum-free DMEM supplemented with 0.005% of vitamin C and 1% of BSA for 24 hours) was loaded into the lower compartment. Cells migrated into the lower compartments of the transwell were counted three times at 24-hour intervals, and then the results were stastically analyzed. As a control group, Fc peptide was treated instead of the above peptides. The results are shown in FIG. 4.

Referring to FIG. 4, in the test groups treated with the polypeptide of SEQ ID NOs: 3 to 5 and 7 to 8 according to the present invention, the invasion rate of the human breast cancer cells was reduced by about 20% relative to that of a control group treated with the Fc peptide. Taking into consideration that cancer cells from blood vessels invade basement membranes or surrounding connective tissues and then spread to secondary sites, it is anticipated that polypeptides of the present invention can effectively inhibit the metastasis of cancer cells.

### Experimental Example 3

### Tests for Inhibitory Activity Against In Vitro Trans-Endothelial Migration of Cancer Cells

Invasion-inducing medium (the supernatant obtained by incubating NIH/3T3 cells (mouse fibroblast cells) in the serum-free DMEM supplemented with 0.005% of vitamin C and 0.1% of BSA for 16 hours) was loaded in each well of the transwells having an inserts, the pore diameter of which is 8-μm. The wells were divided into 3 groups. In the first group (control group), 5×10⁵ MCF-7 cells were treated with 3 μg of a control peptide consisting of Fc. In the second group and the third group, 5×10⁵ MCF-7 cells were treated with 3 μg of a peptide of SEQ ID NOs: 7 and 8, respectively. After the incubation in 5% CO₂ at 37° C. for 1 hour, each well was treated with 0.1% BSA. Invasion-inducing medium (the supernatant obtained by incubating NIH/3T3 cells (mouse fibroblast cells) in the serum-free DMEM supplemented with 0.005% of vitamin C and 1% of BSA for 24 hours) was loaded into the lower compartment. Cells migrated into the lower compartments of the transwell were counted. The results are shown in FIG. 5.

Referring to FIG. 5, the number of transmigrated MCF-7 cells in the test groups was reduced to about 80% of that in the control group. Taking into consideration that transmigration is essential for migration of cancer cells into organs through blood vessels, it is anticipated that polypeptides of the present invention can effectively inhibit the migration of cancer cells.

### Experimental Example 4

### Tests for Inhibitory Activity Against Adhesion of HUVECs to Extracellular Matrix

Effects of polypeptides of SEQ ID NOs: 3-16 on adhesion of human umbilical vein endothelial cells (HUVECs) to fibronectin were tested.

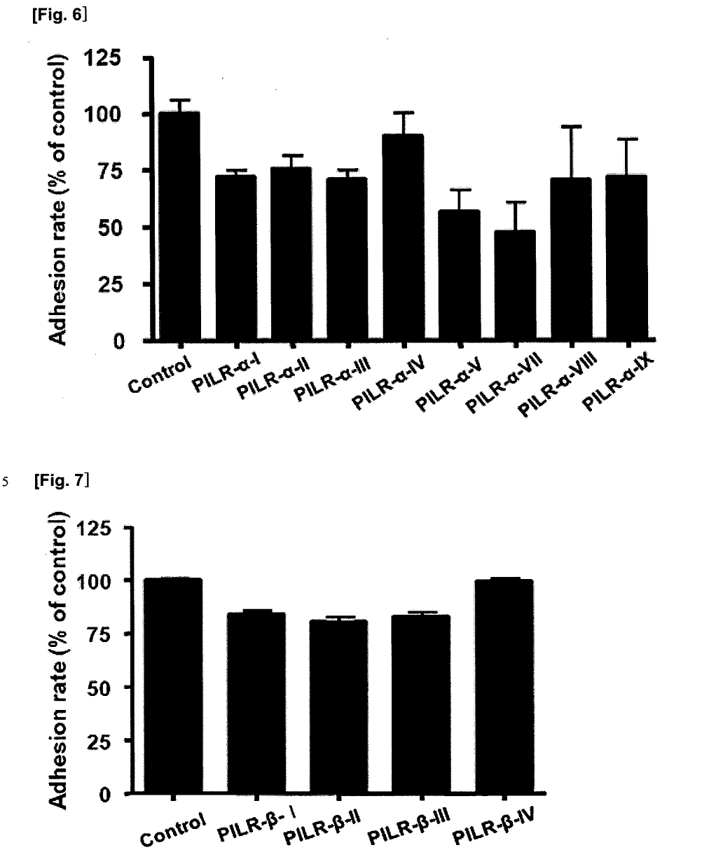

Each well of a 96-well culture plate was streaked with fibronectin, a component of extracellular matrix, and then dried under UV light. HUVECs (5×10⁴) were dispensed into each well, and then the protein solutions including each polypeptide of SEQ ID NOs: 3-16 prepared in Example 2 were treated to each well, in the concentration of 3 μg per each well. After incubation in 5% CO₂ at 37° C. for 1 hour, the cells were washed three times with PBS, detached using trypsin-EDTA, and then stained with a trypan-blue solution. The number of the cells was determined using a hemacytometer. The results are illustrated in FIGS. 6 and 7. In FIGS. 6 and 7, the control peptide is a human IgG Fc, having a polypeptide as set forth in of SEQ ID NO: 18.

Referring to FIGS. 6 and 7, in the test groups treated with the polypeptides of the present invention, the number of HUVECs adhered to fibronectin was reduced by about 10-50% relative to a control group. And also, in case treated with the fusion proteins, i.e., PILRα I, II, and III-Fc (or PILRβ I, II, and III-Fc), similar results were also obtained (data not shown). However, in the test group treated with the polypeptide of SEQ ID NOs: 6 or 15 including no amino acids at positions 123-126 of SEQ ID NOs: 1 and 2, the degree of adhesion of HUVECs to fibronectin was similar to that of the control group. Thus, it is anticipated that polypeptides of the present invention including peptides at positions 123-126 of SEQ ID NOs: 1 or 2 can inhibit angiogenesis.

### Experimental Example 5

### Tests for Inhibitory Activity Against In Vitro Angiogenesis

Effects of polypeptides of the present invention were evaluated on angiogenesis. Generally, interactions of basement membrane components of blood vessels with vascular endothelial cells play an important role in formation and maintenance of new blood vessels. When Matrigel, one of the basement membrane components, is treated to 24-well culture plate containing vascular endothelial growth factor (VEGF), plugs are formed through polymerization reaction.

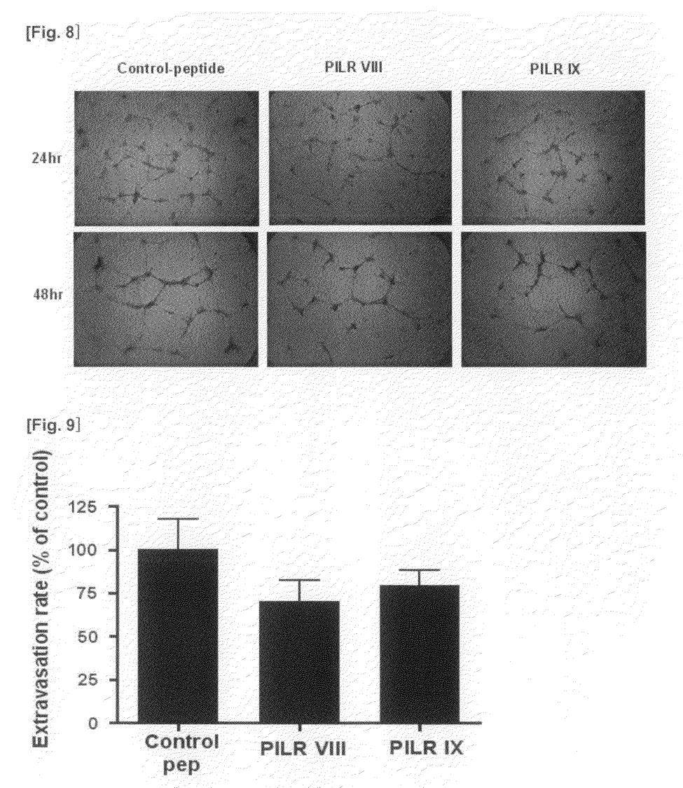

HUVECs were seeded at a density of 8×10⁴ cells/well to each well of 24-well culture plates coated with Matrigel. The protein solutions including each polypeptide of SEQ ID NOs: 10 or 11 (30 μg/Ml) prepared in Example 2 and bFGF (basic fibroblast growth factor, 3 ng/Ml) were added to the wells. After incubation for 24 hours, formation of new blood vessels was examined using an inverted microscope (at 50× magnification), and the results are shown in FIG. 8. In FIG. 8, the control peptide is a peptide consisting of EEFD.

Referring to FIG. 8, when HUVECs were treated with the protein solution including the polypeptide of the present invention, tube formation (i.e., angiogenesis) was significantly reduced.

### Experimental Example 6

### Tests for Inhibitory Activity Against In Vitro Trans-Endothelial Migration of Monocytes

HUVECs were cultured in the upper compartments of Boyden chambers. The supernatants were removed, and human monocytes (U937), which had been untreated or treated with the protein solution including the polypeptide of SEQ ID NOs: 10 or 11 (30 μg/Ml) prepared in Example 2 for 1 hour, were seeded at 5×10⁵ cells/chamber. At this time, a culture including a supernatant obtained by centrifugation of a culture obtained after culturing NIH/3T3 mouse fibroblasts in serum-free DMEM containing 0.005% vitamin C and 0.1% Bovine Serum Albumin (BSA) for 16 hours was placed in the lower compartments of the chambers to induce the invasion of the monocytes. The chambers were incubated for 6 hours, and the number of the cells migrated to the lower compartments was measured. The test was repeated five times, and the results are illustrated in FIG. 9. In FIG. 9, the control peptide is a peptide consisting of EEFD.

Referring to FIG. 9, the number of migrated monocytes in the test groups treated with polypeptides according to the present invention was significantly reduced (about ⅔ reduction) as compared with that in the control group. Taking into consideration that transmigration is essential for migration of leukocytes into inflammation sites through blood vessels, it is anticipated that polypeptides of the present invention can effectively inhibit the inflammatory reaction.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A polypeptide consisting of 4-200 amino acids derived from the peptide of SEQ ID NOs: 1 or 2, wherein said polypeptide comprising an amino acid sequence from position 123 to position 126 of SEQ ID NOs: 1 or 2; and wherein said polypeptide inhibits transmigration of leukocytes or growth and/or metastasis of cancer cells.

2. The polypeptide of claim 1, which is selected from the group consisting of polypeptides as set forth in SEQ ID NOs: 3-5, 7-14, and 16.

3. A fusion protein wherein a polyhistidine (poly-His) region is fused to the polypeptide of claim 1.

4. The fusion protein of claim 3, wherein the poly-His region has an amino acid sequence as set forth in SEQ ID NO: 17.

5. A fusion protein wherein a Fc region is fused to the polypeptide of claim 1.

6. The fusion protein of claim 5, wherein the Fc region has an amino acid sequence as set forth in SEQ ID NO: 18.

7. A polynucleotide encoding the polypeptide of claim 1.

8. The polynucleotide of claim 7, which is selected from the group consisting of polynucleotides as set forth in SEQ ID NOs: 19-21, 23-30, and 32.

9. A vector comprising a polynucleotide encoding the polypeptide of claim 1.

10. The vector of claim 9, wherein a cDNA encoding a poly-His region or a Fc region is inserted.

11. A transformant obtained by transforming a host cell with the vector of claim 9.

12. The transformant of claim 11, wherein the host cell is selected from cells of microorganisms belonging to the genus Escherichia and the genus Pichia.

13. A pharmaceutical composition for inhibiting the growth and/or metastasis of cancer cells, comprising the polypeptide or its fusion protein of claim 1 as an active ingredient and a pharmaceutically acceptable carrier.

14. A pharmaceutical composition for the prevention or treatment of inflammatory diseases, comprising the polypeptide or fusion protein of claim 1 as an active ingredient and a pharmaceutically acceptable carrier.
